(19) United States
(12) Patent Application Publication
(10) Pub. No.: US 2026/0234092 A1
(43) Pub. Date: Aug. 13, 2026

(54) METHOD AND APPARATUS FOR PRODUCING ALDEHYDE

(71) Applicant: JOHNSON MATTHEY DAVY TECHNOLOGIES LIMITED, London (GB)

(72) Inventors: Adrian BACKES, London (GB); Michael WILLIAMS, London (GB)

(21) Appl. No.: 19/154,212

(22) PCT Filed: Mar. 11, 2024
(86) PCT No.: PCT/GB2024/050648

§ 371 Date: Aug. 6, 2025

(30) Foreign Application Priority Data
Mar. 13, 2023 (GB) 2303617.1

(51) Int. Cl. C07C 45/50; B01D 3/14; B01D 5/00; B01J 8/04

(52) U.S. Cl. CPC C07C 45/505; B01D 3/143; B01D 5/0036; B01D 5/006; B01J 8/0492

(57) ABSTRACT

A method for the production of a C₂ₙ unsaturated aldehyde, wherein n is in the range of and including 3 to 6, the method comprising: (iv) passing a crude aldol stream from an aldolisation reaction to a crude aldol distillation column operated under distillation conditions to form a bottom stream having an increased concentration of the C₂ₙ unsaturated aldehyde, water and heavies as compared to the crude aldol stream, and an overhead stream comprising unreacted Cₙ aldehyde, Cₙ₋₁ alkane(s), Cₙ₋₁ alkene(s) and a reduced concentration of the C₂ₙ unsaturated aldehyde, water and heavies as compared to the crude aldol stream; (v) passing the overhead stream from the crude aldol distillation column to a first condenser zone, the first condenser zone being configured to provide a first condensed stream comprising an increased concentration of unreacted Cₙ aldehyde and water as compared to the overhead stream from the crude aldol distillation column.

### FIELD OF INVENTION

The present invention relates to a method and apparatus for the production of aldehydes. In particular, the methods and apparatus according to the present invention provide improved feedstock efficiency in the production of synthetic aldehydes, more particularly 2-alkylalkenals

### BACKGROUND

Synthetic aldehydes, such as 2-alkylalkenals, are generally used as intermediates in the production of alcohols, such as 2-alkylalkanols, which themselves are used in a wide variety of applications in the chemical industries including as solvents, fuels, chemical intermediates in synthesis of organic compounds, and such like. Globally, a wide variety of alcohols are produced each year. For certain alcohols, for example ethanol, biosynthetic methods such as fermentation may be suitable for the production of alcohol. However, for longer chain and branched-chain alcohols, synthetic production is generally required.

Existing methods for the synthetic production of aldehydes such as 2-alkylalkenals include the step of hydroformylation of olefins in the presence of a catalyst, also referred to as the ‘Oxo’ process, followed by aldolisation. Hydroformylation introduces a formyl group to the unsaturated olefin to provide an aldehyde. Hydroformylation may be effected by contacting the olefin with syngas, a mixture of carbon monoxide and hydrogen. Once the aldehyde has been produced, hydrogenation reduces the aldehyde to furnish the corresponding alcohol.

Where longer chain or branched chain alcohols are required, the process may include the step of aldolisation to condense two aldehydes, followed by dehydration, thus providing an unsaturated aldehyde such as a 2-alkylalkenal. Such aldolisation reaction products are typically referred to as α,β-unsaturated aldehydes. Where the aldolisation reaction is between two of the same aldehyde, the α,β-unsaturated aldehyde is referred to as a self-condensation product. Where the aldolisation reaction is between two different aldehydes, the α,β-unsaturated aldehyde is referred to as a cross-condensation product. The aldolisation process is typically base or acid catalysed.

The unsaturated aldehyde is then subsequently reduced by hydrogenation to provide the corresponding saturated alcohol.

Given the wide utility of alcohols, particularly 2-alkylalkanols, it is desirable to produce the alcohols in a cost-efficient manner, on an industrial scale with a maximum yield and with maximum purity.

It is therefore an object of the present invention to improve the feedstock efficiency and yield in the preparation of aldehydes, particularly 2-alkylalkenals, produced by industrial processes. Such an advantage is a benefit in its own right, and also a benefit to the overall feedstock efficiency and yield in the production of alcohols, particularly 2-alkylalkanols, in which the aldehydes are an intermediate.

### SUMMARY OF THE INVENTION

The inventors provide a new method and apparatus for the production of a C₂ₙ unsaturated aldehyde from a Cₙ aldehyde, wherein n is in the range of and including 3 to 6, via an aldolisation reaction, more particularly 2-alkylalkenals, and more particularly still 2-propyl hept-2-enal. Advantageously, the method and apparatus of the present invention increases the yield of the product and increases feedstock efficiency.

The method of the present invention and apparatus enables enhanced recovery of unreacted Cₙ aldehyde from a crude aldol distillation column provided after the aldolisation reaction. The distillation column is configured to separate the reaction mixture provided by the aldolisation step. More particularly, the present invention provides a first condenser zone and a second condenser zone overhead the distillation column to recover a first condensed stream comprising unreacted Cₙ aldehyde, a Cₙ/Cₙ₋₁ condensate comprising the remaining unreacted Cₙ aldehyde, along with Cₙ₋₁ alkane(s) and Cₙ₋₁ alkene(s), and an optional vapour stream comprising Cₙ₋₁ alkane(s) and Cₙ₋₁ alkenes. The Cₙ₋₁ alkane(s) and Cₙ₋₁ alkene(s) are typically present in the Cₙ aldehyde feed stream to the aldolisation reaction because they are present in the feed to the hydroformylation reaction. A small amount of the Cₙ₋₁ alkane(s) and Cₙ₋₁ alkene(s) are typically allowed to slip through to the aldolisation reaction to moderate the temperature in the base of a crude aldehyde distillation column which is described herein and used to at least partially separate Cₙ₋₁ alkane(s) and Cₙ₋₁ alkene(s) from the Cₙ aldehyde. The Cₙ aldehyde, Cₙ₋₁ alkane(s) and Cₙ₋₁ alkene(s) accumulate at the top of the aldol distillation column and concentrate to a high level due to their hydrophobic nature, and are conventionally vented to limit their build up. Typically, this has been achieved by partially condensing the overheads from the crude aldol distillation column in a single step of condensing, for example by cooling to around 80° C. in the case of pentanal used in the synthesis of 2-propyl hept-2-enal. This leads to loss of feedstock Cₙ aldehyde from the process.

According to a first aspect of the invention, there is provided a method for the production of a C₂ₙ unsaturated aldehyde, wherein n is in the range of and including 3 to 6, the method comprising:
(i) feeding an olefin stream comprising a Cₙ₋₁ alkene to a hydroformylation reactor, and contacting the olefin stream with a hydroformylation gas stream comprising hydrogen and carbon monoxide to effect a hydroformylation reaction to provide a crude aldehyde stream comprising a Cₙ aldehyde, Cₙ₋₁ alkane(s), and Cₙ₋₁ alkene(s);
(ii) feeding the crude aldehyde stream to a crude aldehyde distillation zone operated under distillation conditions to form a bottom stream comprising the Cₙ aldehyde, Cₙ₋₁ alkane(s), and Cₙ₋₁ alkene(s) and comprising an increased concentration of the Cₙ aldehyde relative to the crude aldehyde stream and an overhead stream comprising an increased concentration of Cₙ₋₁ alkane(s), and Cₙ₋₁ alkene(s) relative to the crude aldehyde stream;
(iii) feeding the bottom stream from the crude aldehyde distillation column to an aldolisation reactor under condensation and dehydration conditions to effect an aldolisation reaction of the Cₙ aldehyde to provide a crude aldol stream comprising an aldol product C₂ₙ unsaturated aldehyde, unreacted Cₙ aldehyde, Cₙ₋₁ alkane(s), Cₙ₋₁ alkene(s), water and heavies;
(iv) passing the crude aldol stream to a crude aldol distillation column operated under distillation conditions to form a bottom stream having an increased concentration of the C₂ₙ unsaturated aldehyde, water and heavies as compared to the crude aldol stream, and an overhead stream comprising unreacted Cₙ aldehyde, Cₙ₋₁ alkane(s), Cₙ₋₁ alkene(s) and a reduced concentration of the C₂ₙ unsaturated aldehyde, water and heavies as compared to the crude aldol stream;
(v) passing the overhead stream from the crude aldol distillation column to a first condenser zone, the first condenser zone being configured to provide a first condensed stream comprising an increased concentration of unreacted Cₙ aldehyde and water as compared to the overhead stream from the crude aldol distillation column and an overhead stream comprising Cₙ₋₁ alkane(s), Cₙ₋₁ alkene(s) and a reduced concentration of unreacted Cₙ aldehyde and water as compared to the overhead stream from the crude aldol distillation column;
(vi) passing the overhead stream from the first condenser zone to a second condenser zone, the second condenser zone being configured to condense at least a portion of the overhead stream from the first condenser zone to provide Cₙ/Cₙ₋₁ condensate; and
wherein at least a portion of the Cₙ/Cₙ₋₁ condensate is recovered and returned to upstream of the crude aldehyde distillation column or to the crude aldehyde distillation column.

Advantageously, the crude aldehyde distillation zone is more effective at separating Cₙ aldehyde from Cₙ₋₁ alkane(s) and Cₙ₋₁ alkene(s) and facilitates efficient recycling of the unreacted Cₙ aldehyde back to the aldolisation step rather than wasting it to vent. Recovery of the Cₙ₋₁ alkane(s) and Cₙ₋₁ alkene(s) in the crude aldehyde distillation zone additionally allows the stream to be exported as a liquid rather than a low-pressure gas, thereby increasing the value of the recovered Cₙ₋₁ alkane(s) and Cₙ₋₁ alkene(s) stream.

In the method of the invention, n is suitably 4, 5 or 6, typically 4 or 5, preferably 5. Accordingly, The Cₙ₋₁ alkene in the olefin feed stream may suitably be propene or butene, preferably butene, for example but-1-ene. The Cₙ aldehyde is suitably a C₄, C₅ or C₆ aldehyde, typically C₄ or C₅, preferably C₅, i.e. pentanal. The majority of the Cₙ aldehyde which undergoes the aldolisation reaction to provide the aldol product C₂ₙ unsaturated aldehyde is linear aldehyde, e.g. valeraldehyde (pentanal). Typically, the branched aldehyde will be present in an amount of at most 10 mol %, suitably at most 7.5 mol % of the total Cₙ aldehyde. For example 3-methylbutanal and 2-methylbutanal may be present where the Cₙ aldehyde is pentanal. Such isomers may typically be generated during the hydroformylation step. The C₂ₙ unsaturated aldehyde is suitably a C₈, C₁₀ or C₁₂ unsaturated aldehyde, typically a C₈ or C₁₀ unsaturated aldehyde, preferably C₁₀. The C₂ₙ unsaturated aldehyde may be a 2-alkylalkenal. Preferably, the C₂ₙ aldehyde is 2-propyl hept-2-enal.

The Cₙ₋₁ alkane(s) may be a Cₙ₋₁ alkane, or a mixture of isomeric Cₙ₋₁ alkanes. For example, in the preferred aspect in which n is 5 and the Cₙ aldehyde is pentanal, the Cₙ₋₁ alkane(s) include n-butane and i-butane, typically n-butane. The Cₙ₋₁ alkane(s) present in the method of the invention may be carried through from the olefin feed to the hydroformylation step and/or may be produced during hydroformylation. The Cₙ₋₁ alkene(s) may be a Cₙ₋₁ alkene, or a mixture of isomeric Cₙ₋₁ alkenes. For example, in the preferred aspect in which n is 5 and the Cₙ aldehyde is pentanal, the Cₙ₋₁ alkene(s) include but-1-ene and cis- and trans-but-2-ene. The Cₙ₋₁ alkene(s) present in the method of the invention are typically carried through from the olefin feed to the hydroformylation step, and may also form at various stages throughout the process. The amount of Cₙ₋₁ alkane(s) and Cₙ₋₁ alkene(s) present in the method of the invention will typically depend on the nature of the olefin feed in the hydroformylation step. Other alkane(s), such as Cₙ alkane(s), may also be present which will follow the Cₙ₋₁ alkane(s) and Cₙ₋₁ alkene(s) through the process of the invention. Put another way, a stream comprising an increased concentration of Cₙ₋₁ alkane(s) and Cₙ₋₁ alkene(s) will also have an increased concentration of Cₙ alkane(s). Any Cₙ alkane(s) present in the method of the invention may be carried through from the olefin feed to the hydroformylation step and/or may be produced during hydroformylation.

The amount of the Cₙ₋₁ alkene olefin in the olefin stream is not particularly limited and is not critical to the invention. For example, a suitable olefin stream to the hydroformylation reactor may comprise at least 50 mol % of the Cₙ₋₁ alkene, optionally at least 60 mol % of the Cₙ₋₁ alkene, optionally at least 70 mol % of the Cₙ₋₁ alkene, optionally still at least 80 mol % of the Cₙ₋₁ alkene, further optionally at least 90 mol % of the Cₙ₋₁ alkene, relative to the total moles of the olefin stream.

The hydroformylation reactor may suitably be operated at a single pass conversion of the Cₙ₋₁ alkene of at least 40%, optionally at least 50%, optionally still at least 60%. The concentration of Cₙ aldehyde in the crude aldehyde stream will depend on this conversion level, and is not critical to the invention. The crude aldehyde stream may comprise, e.g., at least 25 mol % of the Cₙ aldehyde relative to the total moles of crude aldehyde stream.

The bottom stream from the crude aldehyde distillation column, which also provides the feed to the aldolisation reactor, may comprise, e.g., at least 70 mol % of the Cₙ aldehyde, further optionally at least 80 mol % of the Cₙ aldehyde, typically at least 90 mol % of the Cₙ aldehyde relative to the total moles of the feed stream.

The overhead stream from the crude aldehyde distillation column may comprise, e.g., at least 95 mol % Cₙ₋₁ alkane(s) and Cₙ₋₁ alkene(s) relative to the total moles of the overhead stream from the crude aldehyde distillation column. This stream can be exported as a liquid rather than a low-pressure gas, thereby increasing the value of the recovered Cₙ₋₁ alkane(s) and Cₙ₋₁ alkene(s) stream.

The aldolisation reaction may suitably be operated at a single pass conversion of the Cₙ aldehyde of at least 50%, optionally at least 60%, optionally still at least 70%. The concentration of C₂ₙ unsaturated aldehyde in the crude aldol stream will depend on this conversion level, and is not critical to the invention. Typically, the crude aldol stream may comprise, e.g., at least 5 mol % of the C₂ₙ unsaturated aldehyde relative to the total moles of the crude aldol stream. A large amount of water may be present in this stream, e.g., at least 50 mol % relative to the total moles of the crude aldol stream. The crude aldol stream will also comprise heavies. As used herein, the term “heavies” refers to organic molecules having a molecular weight greater than the C₂ₙ unsaturated aldehyde and/or a boiling point greater than the C₂ₙ unsaturated aldehyde. The crude aldol stream will also comprise unreacted Cₙ aldehyde along with the Cₙ₋₁ alkane(s) and Cₙ₋₁ alkene(s).

The bottom stream from the crude aldol distillation column may also comprise an aqueous phase, e.g., at least 80 mol % of the bottom stream may be aqueous phase. The aqueous phase, for example when a homogeneous catalyst is used, typically comprises dissolved aldolisation reaction catalyst, e.g. NaOH. The aqueous phase is separated from the organic phase comprising the C₂ₙ unsaturated aldehyde typically by decanting. Alternatively, there may be no aqueous phase to separate, for example when a heterogenous catalyst is used in the aldolisation reaction, and the entire bottom stream is an organic phase. The organic phase provides the feed for the method for the production of a C₂ₙ alcohol described herein. Said method comprises producing a C₂ₙ unsaturated aldehyde according to the method of the invention, then a step (vii) of feeding the organic phase from the bottom stream from the crude aldol distillation column to a hydrogenation zone to effect a hydrogenation of the C₂ₙ unsaturated aldehyde to provide the C₂ₙ alcohol.

The overhead stream from the crude aldol distillation column may comprise, e.g., at least 15 mol % of combined Cₙ₋₁ alkane(s), Cₙ₋₁ alkene(s) and unreacted Cₙ aldehyde relative to the total moles of the overhead stream from the crude aldol distillation column. The overhead stream from the crude aldol distillation column may comprise at least 50 mol % water relative to the total moles of the overhead stream from the crude aldol distillation column. The overhead stream from the crude aldol distillation column may comprise at least 10 mol % of unreacted Cₙ aldehyde relative to the total moles of the overhead stream from the crude aldol distillation column. The overhead stream from the crude aldol distillation column may also comprise minor amounts of the C₂ₙ unsaturated aldehyde, e.g., less than 2 mol %, typically less than 1 mol %.

The first condenser zone typically uses a heat exchanger to condense the overhead stream from the first condenser zone. Preferably, at least a portion of (i.e. not all), or substantially all of, the first condensed stream is recovered as a first condensed stream recycle stream and returned to the aldolisation reactor. The first condensed stream typically comprises water and the first condensed stream is typically passed through a decanter to remove water before being returned to the aldolisation reactor. In the decanter, an aqueous stream is separated from the first condensed stream, to provide the first condensed stream recycle stream. The aqueous stream typically comprises at least 95 mol % water relative to the total moles of the aqueous stream. After passing through the decanter, the first condensed stream recycle stream may comprise, e.g., at least 60 mol % combined Cₙ₋₁ alkane(s), Cₙ₋₁ alkene(s), and unreacted Cₙ aldehyde optionally at least 80 mol % combined Cₙ₋₁ alkane(s), Cₙ₋₁ alkene(s), and unreacted Cₙ aldehyde relative to the total moles of the first condensed stream, and optionally less than 5 mol % water, relative to the total moles of the first condensed stream. The first condensed stream may also comprise any minor remaining amounts of the C₂ₙ unsaturated aldehyde.

The overhead stream from the first condenser zone may typically comprise, e.g., at least 50 mol % combined Cₙ₋₁ alkane(s), Cₙ₋₁ alkene(s), and unreacted Cₙ aldehyde relative to the total moles of the overhead stream from the first condenser zone. The overhead stream from the first condenser zone may comprise, e.g., at least 10 mol % Cₙ aldehyde relative to the total moles of overhead stream from the first condenser zone. The overhead stream from the first condenser zone may also comprise residual water.

In the second condenser zone, Cₙ/Cₙ₋₁ condensate may be produced in a single condensation step or multiple condensation steps. In each condensation step, or the condensation step, the overhead stream from the first condenser zone may be condensed using a heat exchanger. Alternatively or additionally, in each condensation step, or the condensation step, the overhead stream from the first condenser zone may be condensed by washing with a process stream which is cooler than the overhead stream from the first condenser zone. Such a process stream may be actively cooled, if required, such that it is cooler than the overhead stream from the first condenser zone. A suitable process stream is at least a portion of the first condensed stream recycle stream. In this case, the remaining portion of the first condensed stream recycle stream may still be returned to the aldolisation reactor. Another suitable process stream is a portion of the crude aldehyde stream. In this case, the remaining portion of the crude aldehyde stream is still fed to the crude aldehyde distillation zone. Another suitable process stream is a portion of the bottom stream from the crude aldehyde distillation column. In this case the remaining portion of the bottom stream from the crude aldehyde distillation column is still fed to the aldolisation reactor. Such washes further improve the recovery of any remaining Cₙ aldehyde and increase feedstock efficiency.

A portion, i.e. not all, or substantially all of the overhead stream from the first condenser zone may be condensed. In the alternative in which not all of the stream is condensed a vapour stream will be produced by the second condenser zone, which may be vented. This vapour stream suitably comprises an increased concentration of Cₙ₋₁ alkane(s) and Cₙ₋₁ alkene(s) as compared to the overhead stream from the first condenser zone. This vapour stream may comprise, e.g., at least 90 mol % Cₙ₋₁ alkane(s) and Cₙ₋₁ alkene(s), optionally at least 95 mol % Cₙ₋₁ alkane(s) and Cₙ₋₁ alkene(s) relative to the total moles of vapour stream. At least a portion of (i.e. not all) or substantially all of the Cₙ/Cₙ₋₁ condensate is recovered and returned to upstream of the crude aldehyde distillation column or to the crude aldehyde distillation column. The Cₙ/Cₙ₋₁ condensate may typically comprise, e.g., at least 50 mol % combined Cₙ₋₁ alkane(s), Cₙ₋₁ alkene(s), and Cₙ aldehyde relative to the total moles of the Cₙ/Cₙ₋₁ condensate. The Cₙ/Cₙ₋₁ condensate may comprise, e.g., at least 15 mol % Cₙ aldehyde relative to the total moles of the Cₙ/Cₙ₋₁ condensate. The Cₙ/Cₙ₋₁ condensate may also comprise residual water. In that case, a decanter may be used to remove water. In the decanter, an aqueous stream is separated from the Cₙ/Cₙ₋₁ condensate. Typically, at least 20 mol %, optionally at least 50 mol %, further optionally at least 90 mol % of the Cₙ/Cₙ₋₁ condensate is recovered as a recycle stream. Typically, at least 95 mol % of the Cₙ/Cₙ₋₁ condensate is returned to upstream of the crude aldehyde distillation column or to the crude aldehyde distillation column. Advantageously, this recycle can be performed without the need for expensive compression, otherwise necessary because the crude aldol distillation column typically operates at a lower pressure than the crude aldehyde distillation column, because the recycle is provided as a liquid.

The temperature of the first condenser zone will typically be greater than the condensation temperature in the second condenser zone, e.g. at least 10° C. greater. The temperature of the first condenser zone may typically be from 50° C. to 110° C., optionally from 70° C. to 90° C. The temperature of the condensation in the second condenser zone may typically be from 2° C. to 50° C., optionally from 35° C. to 45° C., further optionally from 35° C. to 40° C.

According to a second aspect of the invention, there is provided an apparatus for the production of a C₂ₙ unsaturated aldehyde, wherein n is in the range of 3 to 6, the apparatus comprising:
a hydroformylation reactor configured to contact an olefin stream comprising an alkene with hydrogen and carbon monoxide to effect a hydroformylation reaction on the alkene to provide the feed stream comprising a Cₙ aldehyde, Cₙ₋₁ alkane(s), and Cₙ₋₁ alkene(s);
a crude aldehyde distillation column configured to receive the crude aldehyde stream and provide an overhead stream and a bottom stream;
an aldolisation reactor configured to receive the bottom stream from the crude aldehyde distillation column and to provide a crude aldol stream, wherein the crude aldol stream comprises an aldol product C₂ₙ unsaturated aldehyde, unreacted Cₙ aldehyde, Cₙ₋₁ alkane(s), Cₙ₋₁ alkene(s), water and heavies;
a crude aldol distillation column configured to receive the crude aldol stream and to provide an overhead stream and a bottom stream;
a first condenser zone configured to cool the first overhead stream to provide a first condensed stream and an overhead stream;
a second condenser zone in fluid communication with the first condenser zone, wherein the second condenser zone is configured to cool the first overhead stream to provide a condensate and optionally a vapour stream;
wherein the second condenser zone is in fluid communication with the crude aldehyde distillation column or upstream of the crude aldehyde distillation column to return at least a portion of the condensate to the crude aldehyde distillation column or upstream of the crude aldehyde distillation column;
wherein the first condenser zone is optionally in fluid communication with the aldolisation reactor to return at least a portion of the first condensed stream to the aldolisation reactor.

### BRIEF DESCRIPTION OF THE FIGURES

FIG. 1 illustrates a schematic of a conventional process for the industrial production of aldehydes.

FIG. 2 illustrates a schematic process for the industrial production of aldehydes according to the invention.

FIG. 3 illustrates a schematic process for the industrial production of aldehydes according to the invention.

FIG. 4 illustrates a schematic process for the industrial production of aldehydes according to the invention.

### DETAILED DESCRIPTION

The present invention will now be described in detail with reference to the following non-limiting examples and accompanying figures. Whilst the present invention is generally described with reference to production of 2-propyl hept-2-enal, those skilled in the art will appreciate that the present invention may be applied to the production of any other C₂ₙ unsaturated aldehyde.

2-alkylalkanois as described herein are preferably formed via hydrogenation of an α,β-unsaturated aldehyde, wherein the α,β-unsaturated aldehyde is a self-condensation product. Preferably, the α,β-unsaturated aldehyde is a self-condensation product of pentanal. Preferably, the α,β-unsaturated aldehyde is 2-propyl hept-2-enal.

Scheme 1 below shows a general process for the production of 2-propyl hept-2-enal and 2-propyl heptanol.

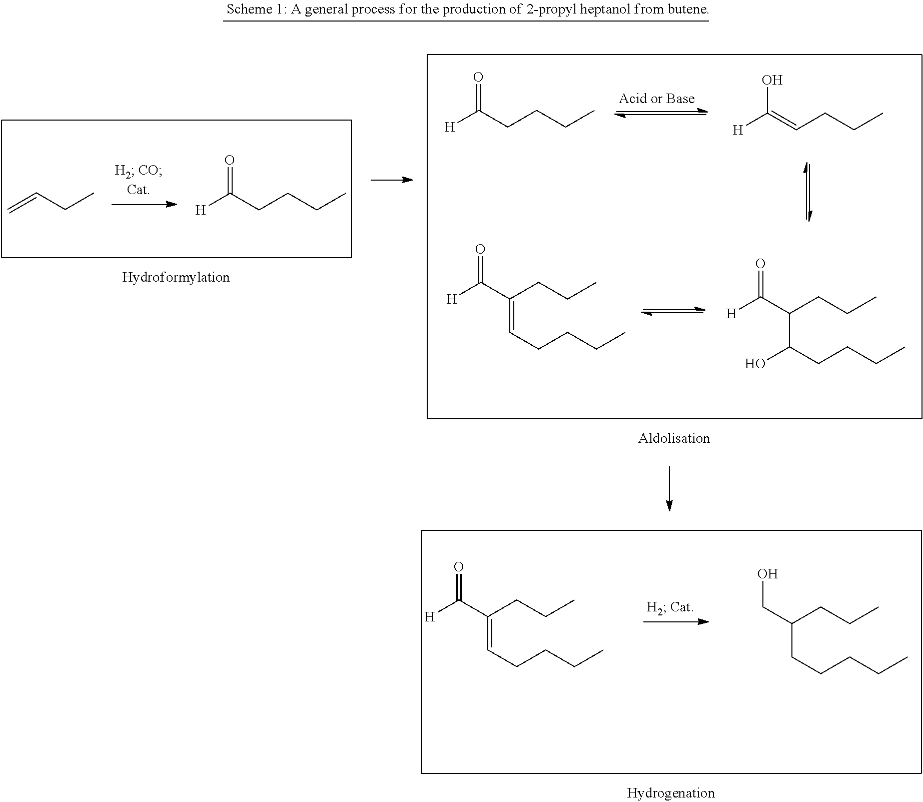

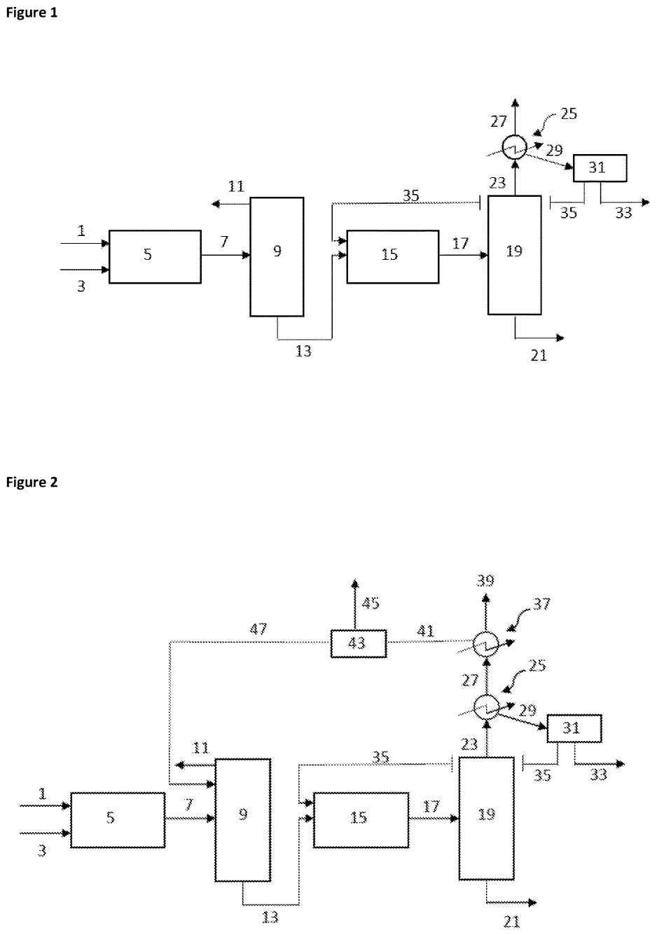

The process for the production of 2-propyl hept-2-enal and 2-propyl heptanol will now be described with reference to Scheme 1 above, and FIG. 1.

With reference to FIG. 1, an olefin stream 1 comprising the Cₙ₋₁ alkene, for example but-1-ene, as well as the Cₙ₋₁ alkane(s), for example isomers of butane, and Cₙ₋₁ alkene(s), for example but-1-ene, cis-but-2-ene and trans-but-2-ene, is provided to a hydroformylation reactor 5. The olefin stream 1 may be provided as a crude cracked product stream from the industrial cracking of hydrocarbons. Those skilled in the art will be familiar with industrial cracking processes. The crude cracked product stream may undergo one or more enrichment steps, for instance by passing the crude cracked product stream through one or more distillation columns to increase the concentration of Cₙ₋₁ alkene in the olefin stream 1 that is provided to the hydroformylation reactor 5.

In the hydroformylation reactor 5, the Cₙ₋₁ alkene is contacted with carbon monoxide and hydrogen, provided via syngas stream 3, in the presence of a liquid catalyst solution to provide a Cₙ aldehyde, and more particularly pentanal. This hydroformylation process is also known as the “oxo process”. Typical flowsheets are described, for example, in U.S. Pat. No. 4,148,830 or U.S. Pat. No. 5,087,763 which are incorporated herein by reference. The hydroformylation liquid catalyst system typically comprises a solvent, rhodium, a ligand and other components, but may be any suitable catalyst system for hydroformylation. Typical organophosphine and organophosphite ligands are described in, for example, WO2008/115740, WO2011/087690, WO2010/117391 and WO2016/089602 which are incorporated herein by reference.

Hydroformylation provides a crude aldehyde stream 7 comprising a mixture of Cₙ₋₁ alkane(s), Cₙ₋₁ alkene(s) and the Cₙ aldehyde hydroformylation product, for example pentanal and isomers 2-methylbutanal and 3-methylbutanal in the case of pentanal.

The crude aldehyde stream 7 is passed to a crude aldehyde distillation zone 9 to increase the concentration of Cₙ aldehyde in the aldolisation reactor feed stream 13 that is provided to the aldolisation reactor 15. The distillation zone 9 may be a single distillation column, or a multi-column distillation set-up, providing that a bottom stream 13 comprising the Cₙ aldehyde, Cₙ₋₁ alkane(s), and Cₙ₋₁ alkene(s) with an increased concentration of Cₙ aldehyde relative to the crude aldehyde stream is produced along with an overhead stream 11 comprising an increased concentration of Cₙ₋₁ alkane(s), and Cₙ₋₁ alkene(s) relative to the crude aldehyde stream. Stream 11 can be exported as a liquid rather than a low-pressure gas, thereby increasing the value of the recovered Cₙ₋₁ alkane(s) and Cₙ₋₁ alkene(s) stream. Additionally some of the stream can be recycled to hydroformylation to convert additional Cₙ aldehyde.

Any suitable means may be used in the distillation zone, and a skilled person can determine, for example, what column internals to use and conditions suitable for the desired separation. A further enrichment step may be included between the crude aldehyde distillation column 9 and the aldolisation reactor 15, but is not essential. For example, a distillation column to increase the amount of linear aldehyde.

The stream 13 is provided to an aldolisation reactor 15. The aldolisation reaction is carried out in aldolisation reactor 15 under condensation and dehydration conditions to effect an aldolisation reaction of the Cₙ aldehyde, for example pentanal, to provide a crude aldol stream comprising an aldol product C₂ₙ unsaturated aldehyde, for example 2-propyl hept-2-enal, along with unreacted Cₙ aldehyde, Cₙ₋₁ alkane(s), Cₙ₋₁ alkene(s), water and heavies. Heavies include trace amounts of the aldolisation reaction catalyst, for example sodium hydroxide, and salts, such as sodium valerate.

Such aldolisation steps are known in the art and described in, for example, U.S. Pat. Nos. 5,434,313 and 6,340,778 which are incorporated herein by reference. Reagents suitable for effecting the aldolisation reaction in the aldolisation reactor 15 include a suitable base or acid. The aldolisation reaction is preferably performed in the liquid phase, that is to say the feed, and the reagents are in the liquid phase. Those skilled in the art will be aware of such suitable acids and bases for the liquid phase reaction, but may be selected from inorganic bases (e.g. NaOH), organic bases (e.g. NEt₃), inorganic acids (e.g. H₂SO₄) and organic acids (e.g. F₃CCOOH). Alternatively, a heterogeneous catalyst may be used and those skilled in the art will be aware of suitable such catalysts. During the aldolisation reaction, water is produced as a by-product (dehydration). To effect the aldolisation reaction, the aldolisation reactor 15 is typically maintained at a temperature of from 80° C. to 140° C. Those skilled in the art are able to determine conditions suitable to operate the aldolisation reaction to effect the desired single-pass conversion of Cₙ aldehyde.

The aldolisation reaction provides a crude aldol stream 17 comprising C₂ₙ unsaturated aldehyde, for example 2-propyl hept-2-enal, unreacted Cₙ aldehyde, Cₙ₋₁ alkane(s), Cₙ₋₁ alkene(s), water and heavies. The aldolisation reaction is typically operated continuously such that the crude aldol stream 17 is removed from the aldolisation reactor 15 during the aldolisation reaction.

The crude aldol stream 17 undergoes an enrichment step by passing the crude aldol stream 17 to a crude aldol distillation column 19 to increase the concentration of C₂ₙ unsaturated aldehyde in the bottom stream 21 that may be decanted to remove water before the organic phase is provided to a hydrogenation reactor (not shown) to produce a C₂ₙ alcohol, for example 2-propyl heptanol.

More particularly, the aldol distillation step comprises passing the crude aldol stream 17 to a crude aldol distillation column 19 operated under distillation conditions to form a bottom stream 21 having an increased concentration of the C₂ₙ unsaturated aldehyde, water and heavies as compared to the crude aldol stream 17, and an overhead stream 23 comprising unreacted Cₙ aldehyde, Cₙ₋₁ alkane(s), Cₙ₋₁ alkene(s) and a reduced concentration of the C₂ₙ unsaturated aldehyde, water and heavies as compared to the crude aldol stream 9. Any suitable means may be used for the distillation, and a skilled person can determine, for example, what column internals to use and conditions suitable for the desired separation. Distillations are described in, for example, U.S. Pat. No. 5,434,313, which is incorporated herein by reference.

FIG. 1 illustrates a crude aldol distillation column 19 comprised in a conventional industrial process for the production of 2-propyl hept-2-enal. Overhead stream 23 is passed to the condenser zone 25, which is configured to provide a condensed stream 29 comprising an increased concentration of Cₙ aldehyde and water as compared to the overhead stream 23 and a vapour stream 27 which contains predominantly Cₙ₋₁ alkane(s) and Cₙ₋₁ alkene(s). The condensed stream 29 also contains Cₙ₋₁ alkane(s) and Cₙ₋₁ alkene(s) as well as any C₂ₙ unsaturated aldehyde that was present in the first overhead stream. The condenser zone 25 comprises a heat exchanger for cooling the overhead stream 23 to a temperature from 70° C. to 90° C., preferably to around 80° C. particularly in the case of the production of 2-propyl hept-2-enal from pentanal. This stream 29 is passed through a decanter 31 which separates out an aqueous stream 33 and a recycle stream 35 which recycles the organics back to aldolisation reactor 15. To alleviate build-up of pressure inside the crude aldol distillation column 19, the vapour stream 27 is simply purged during the distillation process. Vapour stream 27 typically contains some Cₙ aldehyde as well as Cₙ₋₁ alkane(s) and Cₙ₋₁ alkene(s), thus Cₙ aldehyde is lost from the process in the purge.

FIG. 2 is a schematic of a method according to the invention in which there are first 25 and second 37 condenser zones overhead the crude aldol distillation column.

The first condenser zone 25 comprises a first heat exchanger. Overhead stream 23 from the crude aldol distillation column is cooled in the heat exchanger for example by means of a cool water stream. At least a portion of the first condensed stream 29 is decanted and recycled to the aldolisation reactor 15 as per the FIG. 1 method. Typically, the first overhead stream 23 is cooled in heat exchanger 25 to a temperature from 70° C. to 90° C., preferably to around 80° C. particularly in the case of the production of 2-propyl hept-2-enal from pentanal.

Instead of venting in accordance with the FIG. 1 conventional process, the overhead stream 27 from the first condenser zone is sent to a second condenser zone 37. The second condenser zone is configured to provide a Cₙ/Cₙ₋₁ condensate stream 41 comprising an increased concentration of the Cₙ aldehyde, for example pentanal, as compared to the second overhead stream 27. Optionally, a vapour stream 39 is also provided comprising an increased concentration of Cₙ₋₁ alkane(s) and Cₙ₋₁ alkene(s), for example isomers of butane along with but-1-ene, cis-but-2-ene and trans-but-2-ene, as compared to the second overhead stream 27.

The second condenser zone comprises a heat exchanger to provide the Cₙ/Cₙ₋₁ condensate stream 41. The second overhead stream 27 is cooled in the second heat exchanger typically by means of cool water stream. Preferably, the second overhead stream 41 is cooled in the second heat exchanger 37 to around 2° C. to 50° C., optionally from 35° C. to 45° C., further optionally from 35° C. to 40° C., for example to around 38° C. particularly in the case of the production of 2-propyl hept-2-enal from pentanal. The Cₙ/Cₙ₋₁ condensate 41 is passed through a decanter 43 which separates out an aqueous stream 45 and a recycle stream 47 which recycles the organics back to the crude aldehyde distillation column 9. This distillation column is more effective at separating Cₙ aldehyde from Cₙ₋₁ alkane(s) and Cₙ₋₁ alkene(s) and facilitates efficient recycling of the unreacted Cₙ aldehyde back to the aldolisation step, rather than wasting it to vent. Recovery of the Cₙ₋₁ alkane(s) and Cₙ₋₁ alkene(s) from the crude aldehyde distillation column additionally allows the stream to be exported as a liquid rather than a low-pressure gas, thereby increasing the value of the recovered Cₙ₋₁ alkane(s) and Cₙ₋₁ alkene(s) stream.

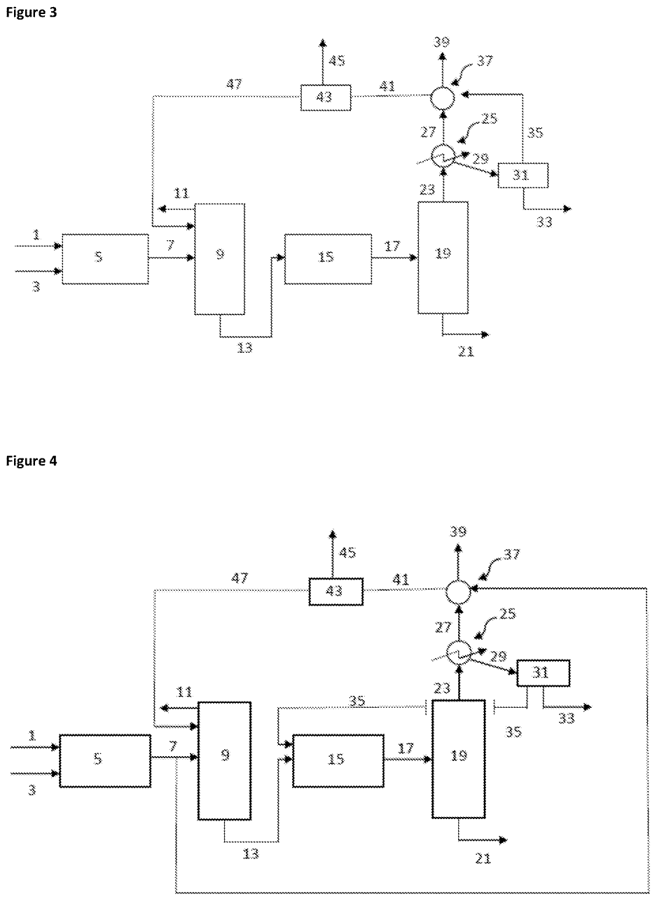

FIG. 3 is a schematic of another method according to the invention in which there are first 25 and second 37 condenser zones overhead the crude aldol distillation column. In this method, recycle stream 35, which is cooler than overhead stream 27 is used to wash overhead stream 27 and condense the stream to provide Cₙ/Cₙ₋₁ condensate stream 41 rather than a heat exchanger. Stream 41 is passed through decanter 43 to provide recycle stream 47 which recycles organics back to the crude aldehyde distillation column 9. Vapour stream 39 containing Cₙ₋₁ alkane(s) and Cₙ₋₁ alkene(s) is optional.

FIG. 4 is a schematic of another method according to the invention in which there are first 25 and second 37 condenser zones overhead the crude aldol distillation column. In this method, a portion of crude aldehyde stream 7 is used to wash overhead stream 27 and condense the stream to provide Cₙ/Cₙ₋₁ condensate stream 41 rather than a heat exchanger. If required, this wash stream is cooled in a cooling stage (not shown). Stream 41 is passed through decanter 43 to provide recycle stream 47 which recycles organics back to the crude aldehyde distillation column 9. Vapour stream 39 containing Cₙ₋₁ alkane(s) and Cₙ₋₁ alkene(s) is optional. A portion of crude aldol stream 7 is still fed to crude aldehyde distillation zone 9.

The bottom stream 21 from the crude aldol distillation column in FIGS. 1 to 4 which comprises an increased concentration of C₂ₙ unsaturated aldehyde, for example 2-propyl hept-2-enal, may then be decanted to remove the aqueous phase before the organic phase is provided to a hydrogenation reactor (not shown). Under hydrogenation conditions in the reactor in the presence of hydrogen and a catalyst, the C₂ₙ unsaturated aldehyde undergoes hydrogenation to provide a C₂ₙ alcohol, for example 2-propyl heptanol. Suitable hydrogenation processes are known in the art and disclosed in, for example, WO2018/069714 which is incorporated herein by reference. The hydrogenation reactor may be operated under any suitable conditions. A catalyst will generally be used. Any suitable catalyst may be used. Generally the active component of the catalyst will be based on metals from Groups VI to X. Suitable examples include copper, nickel, manganese, zinc, cobalt, palladium, ruthenium and iron. The catalyst may be supported. Any suitable support may be used. Suitable supports include alumina, silica or diatomaceous earth. A particularly suitable catalyst may be a supported copper chromite catalyst. The catalyst may also include a promoter to enhance selectivity.

The hydrogenation may be carried out in the liquid or vapour phase. Any suitable configuration may be used and the reactor may be operated under any suitable conditions. Whilst the particular conditions selected will depend on the catalyst chosen, the hydrogenation may be carried out at a temperature of from about 100° C. to about 200° C. and at a pressure of from atmospheric to about 15 MPa.

Where a liquid phase hydrogenation is to be used, it may be performed in any suitable manner. In one arrangement it may be carried out as downflow over packed beds of catalyst. A large recycle of cooled product may be mixed with the feed in order to remove the heat of reaction. One example of a suitable process is described in GB1362071 which is incorporated herein by reference. In an alternative arrangement one or more heat exchangers may be used to remove the heat of reaction.

The crude alcohol stream comprising the C₂ₙ alcohol, for example 2-propyl heptanol may then be purified via one or more purification steps, to provide a purified alcohol stream. For instance, the crude alcohol stream may undergo one or more distillation and polishing steps, for example as described in, for example WO2018/069714.

### Examples

The following example demonstrates that the method and apparatus according to the present invention allow for significant recovery of unreacted C₅ aldehyde which would otherwise be waste. The following example utilises a computational model of the system illustrated schematically FIG. 2 using AVEVA PROII simulator and the known properties (e.g. boiling point, density etc.) of known components (e.g. water, butene, pentanal) in each stream, and vapour liquid equilibrium data. The data demonstrates that significant quantities of useful small organic molecules can be recovered from the Cₙ/Cₙ₋₁ condensate stream 41 using the methods and apparatus of the present invention.

The results of the simulation analysis are provided in the table below.

| FIG. 1 | FIG. 2 | |
|---|---|---|
| (Comparative) | Overhead | Cₙ/Cₙ₋₁ |
| Overhead | Stream | condensate |
| Stream 27 | 39 | Stream 41 |

Stream Composition (mol %)

| | | | |
|---|---|---|---|
| H₂O | 26.4 | 3.5 | 1.3 |
| 2-methyl | 10.1 | 1.4 | 18.3 |
| butyraldehyde | | | |
| (2MBH) | | | |
| Pentanal | 14.8 | 1.4 | 27.2 |
| 3-methyl | 0.1 | 0.0 | 0.2 |
| butyraldehyde | | | |
| (3MBH) | | | |
| But-1-ene | 0.0 | 0.1 | 0.0 |
| Cis But-2-ene | 12.6 | 25.1 | 13.5 |
| Trans but-2-ene | 23.5 | 49.1 | 24.2 |
| Pentane | 0.6 | 0.4 | 1.0 |
| Butane | 11.8 | 19.1 | 14.4 |

Stream Properties

| | | | |
|---|---|---|---|
| Mass Flow Rate | 327 | 86 | 214 |
| (Kg/h) | | | |
| Phase | Vapor | Vapor | Liquid |
| Molecular Weight* | 53.9 | 56.1 | 69.8 |
| Pressure (MPa) | 0.18 | 0.17 | 0.17 |
| Temperature (° C.) | 80 | 38 | 38 |

*average molecular weight of the stream based on the mol % of the components in the relevant stream

These data show that the apparatus of FIG. 3 advantageously allows significant recovery of C₅ aldehyde, which can be recycled to the crude aldehyde distillation column and re-enter the aldolisation reaction, with concomitant separation and recovery of C₄ alkanes and alkenes. As demonstrated by the mass flow rate, the majority of the composition of overhead stream 27 in conventional methods is recovered as recyclable organics in Cₙ/Cₙ₋₁ condensate stream 41 using methods of the invention.

The apparatus of FIG. 2 is calculated to result in approximately a 1% increase in the yield of 2-proyl heptanol after hydrogenation of the produced 2-propyl hept-2-enal, relative to the use of the apparatus in FIG. 1.

The table below shows the effect of temperature of the second heat exchanger in methods and apparatus of the present invention on the mass flow rate of the Cₙ/Cₙ₋₁ condensate stream 41.

| Condensing Temp (° C.) | 36 | 38 | 40 | 42 |
|---|---|---|---|---|
| Mass Flow Rate (kg/h); | 227 | 214 | 202 | 192 |
| Cₙ/Cₙ₋₁ Condensate Stream 41 | | | | |

1. A method for the production of a C₂ₙ unsaturated aldehyde, wherein n is in the range of and including 3 to 6, the method comprising:
(i) feeding an olefin stream comprising a Cₙ₋₁ alkene to a hydroformylation reactor, and contacting the olefin stream with a hydroformylation gas stream comprising hydrogen and carbon monoxide to effect a hydroformylation reaction to provide a crude aldehyde stream comprising a Cₙ aldehyde, Cₙ₋₁ alkane(s), and Cₙ₋₁ alkene(s);
(ii) feeding the crude aldehyde stream to a crude aldehyde distillation zone operated under distillation conditions to form a bottom stream comprising the Cₙ aldehyde, Cₙ₋₁ alkane(s), and Cₙ₋₁ alkene(s) and comprising an increased concentration of the Cₙ aldehyde relative to the crude aldehyde stream and an overhead stream comprising an increased concentration of Cₙ₋₁ alkane(s), and Cₙ₋₁ alkene(s) relative to the crude aldehyde stream;
(iii) feeding the bottom stream from the crude aldehyde distillation column to an aldolisation reactor under condensation and dehydration conditions to effect an aldolisation reaction of the Cₙ aldehyde to provide a crude aldol stream comprising an aldol product C₂ₙ unsaturated aldehyde, unreacted Cₙ aldehyde, Cₙ₋₁ alkane(s), Cₙ₋₁ alkene(s), water and heavies;
(iv) passing the crude aldol stream to a crude aldol distillation column operated under distillation conditions to form a bottom stream having an increased concentration of the C₂ₙ unsaturated aldehyde, water and heavies as compared to the crude aldol stream, and an overhead stream comprising unreacted Cₙ aldehyde, Cₙ₋₁ alkane(s), Cₙ₋₁ alkene(s) and a reduced concentration of the C₂ₙ unsaturated aldehyde, water and heavies as compared to the crude aldol stream;
(v) passing the overhead stream from the crude aldol distillation column to a first condenser zone, the first condenser zone being configured to provide a first condensed stream comprising an increased concentration of unreacted Cₙ aldehyde and water as compared to the overhead stream from the crude aldol distillation column and an overhead stream comprising Cₙ₋₁ alkane(s), Cₙ₋₁ alkene(s) and a reduced concentration of unreacted Cₙ aldehyde and water as compared to the overhead stream from the crude aldol distillation column;
(vi) passing the overhead stream from the first condenser zone to a second condenser zone, the second condenser zone being configured to condense at least a portion of the overhead stream from the first condenser zone to provide Cₙ/Cₙ₋₁ condensate; and
wherein at least a portion of the Cₙ/Cₙ₋₁ condensate is recovered and returned to upstream of the crude aldehyde distillation column or to the crude aldehyde distillation column.

2. The method according to claim 1, wherein at least a portion of the first condensed stream is recovered as a first condensed stream recycle stream and returned to the aldolisation reactor.

3. The method according to claim 1, wherein the second condenser zone is configured to condense substantially all of the overhead stream from the first condenser zone.

4. The method according to claim 1, wherein at least a portion of the overhead stream from the first condenser zone is condensed by washing with a process stream.

5. The method according to claim 1, wherein at least a portion of the overhead stream from the first condenser zone is condensed using a heat exchanger.

6. The method according to claim 1, wherein the temperature of the first condenser zone is greater than the condensation temperature of the second condenser zone.

7. The method according to claim 1, wherein n is 3, 4 or 5.

8. The method according to claim 7, wherein the C₂ₙ unsaturated aldehyde is a 2-alkylalkenal.

9. The method according to claim 8, wherein the C₂ₙ unsaturated aldehyde is 2-propyl hept-2-enal.

10. The method according to claim 1, wherein the Cₙ aldehyde is pentanal.

11. The method according to claim 1, wherein the crude aldehyde distillation zone comprises a single crude aldehyde distillation column operated under distillation conditions to form a bottom stream comprising Cₙ aldehyde, Cₙ₋₁ alkane(s), and Cₙ₋₁ alkene(s) and comprising an increased concentration of Cₙ aldehyde relative to the crude aldehyde stream and an overhead stream comprising an increased concentration of Cₙ₋₁ alkane(s), and Cₙ₋₁ alkene(s) relative to the crude aldehyde stream.

12. A method for the production of a C₂ₙ alcohol, the method comprising producing a C₂ₙ unsaturated aldehyde according to the method of claim 1, then a step (vii) of feeding the organic phase from the bottom stream from the crude aldol distillation column to a hydrogenation zone to effect a hydrogenation of the C₂ₙ unsaturated aldehyde to provide the C₂ₙ alcohol.

13. An apparatus for the production of a C₂ₙ unsaturated aldehyde, wherein n is in the range of 3 to 6, the apparatus comprising:
a hydroformylation reactor configured to contact an olefin stream comprising an alkene with hydrogen and carbon monoxide to effect a hydroformylation reaction on the alkene to provide the feed stream comprising a Cₙ aldehyde, Cₙ₋₁ alkane(s), and Cₙ₋₁ alkene(s);
a crude aldehyde distillation column configured to receive the crude aldehyde stream and provide an overhead stream and a bottom stream;
an aldolisation reactor configured to receive the bottom stream from the crude aldehyde distillation column and to provide a crude aldol stream, wherein the crude aldol stream comprises an aldol product C₂ₙ unsaturated aldehyde, unreacted Cₙ aldehyde, Cₙ₋₁ alkane(s), Cₙ₋₁ alkene(s), water and heavies;
a crude aldol distillation column configured to receive the crude aldol stream and to provide an overhead stream and a bottom stream;
a first condenser zone configured to cool the first overhead stream to provide a first condensed stream and an overhead stream;
a second condenser zone in fluid communication with the first condenser zone, wherein the second condenser zone is configured to cool the first overhead stream to provide a condensate and optionally a vapour stream;
wherein the second condenser zone is in fluid communication with the crude aldehyde distillation column or upstream of the crude aldehyde distillation column to return at least a portion of the condensate to the crude aldehyde distillation column or upstream of the crude aldehyde distillation column;
wherein the first condenser zone is optionally in fluid communication with the aldolisation reactor to return at least a portion of the first condensed stream to the aldolisation reactor.
